(19) United States
(12) Patent Application Publication
(10) Pub. No.: US 2012/0010144 A1
(43) Pub. Date: Jan. 12, 2012

(54) Peg-albumin composition having at least one protected thiol region as a platform for medications

(76) Inventors: Ragheb Assaly, Sylvania, OH (US); John David Digman, Perrysburg, OH (US); Joseph I Shapiro, Toledo, OH (US)

(21) Appl. No.: 13/138,558

(22) PCT Filed: Apr. 2, 2010
(86) PCT No.: PCT/US10/00994

§ 371 Date: Sep. 2, 2011

Related U.S. Application Data
(60) Provisional application No. 61/211,796, filed on Apr. 3, 2009.

(51) Int. Cl. A61K 38/38; A61P 43/00

(52) U.S. Cl. 514/15.2

(57) ABSTRACT

This composition comprises a polyethylene glycol-albumin composition having at least one protected thiol region wherein the composition comprises PEG-AlbCys34 with Cys-34 preserved as a thiol. The albumin is linked to medications such as, antioxidants with a reduced sulfhydryl group through the Cys 34 residing on the albumin. The compositions are useful in treating patients.

### CROSS REFERENCE TO RELATED APPLICATIONS

The present application is based upon and claims the benefit of U.S. provisional patent application No. 61/211,796 filed Apr. 3, 2009.

### FIELD OF THE INVENTION

The present relates to the use of an albumin-based colloid composition, such as PEG-Alb, a polyethylene oxide (such as polyethylene glycol (PEG)) modified albumin, as a platform for medications. More specifically, the platform is for antioxidants and other medications.

### BACKGROUND OF THE INVENTION

A recent development of ours relates to a composition comprising an albumin-based colloid composition. The albumin-based colloid composition is modified such that its hydrodynamic radius is sufficiently large to preclude its leaking through the capillaries while retaining its oncotic properties and its ability to bind ligands such as sodium ions, fatty acids, drugs and bilirubin. A number of proteins have been modified with polyethylene glycol, attached through the ε-amino group of lysine, without loss of biological activity and without significant toxicity. Our use of PEGylation products which expand the composition's hydrodynamic ratio to a degree such that, when administered to a patient suffering from a hypovolemic state, with a danger of developing multiorgan dysfunction (MODS) the albumin-based colloid composition reverses the hypovolemic condition.

The albumin-based colloid composition is especially useful for volume expansion in states of shock such as severe sepsis, shock, pancreatitis, burn and trauma, thereby improving survival rates in those conditions. The composition comprises PEG-Alb_{Cys-34} having a large hydrodynamic radius with Cys-34 preserved as a thiol.

The composition is described in U.S. patent application Ser. No. 10/985,798, filed Nov. 9, 2004, which herein is incorporated by reference.

Albumin as an anti-apoptotic and anti-inflammatory agent. Despite conflicting studies of the clinical efficacy of albumin resuscitation, a number of lines of evidence indicate that albumin maintains the integrity of the vascular endothelium by filling hydrophilic pores of the endothelial surface layer, contributing to their stability. Albumin inhibits endothelial cell apoptosis in human tissues explants in rat skin. Albumin acts as a source of reduced thiols (Cys-34); this effect has been demonstrated in septic patients with increases in thiol concentration of up to 50% following administration of 200 ml 20% albumin.

### SUMMARY OF THE INVENTION

In one embodiment, we link a family of antioxidant medications to the PEG-Alb platform of this invention. The medications are linked with the reduced sulfhydryl group (RSH) through the Cys 34 residing on albumin (Cys-S—SR). Once pegylation of the albumin is performed on lysine residues, the preparation is purified.

We reduce the SH on Cys-34 by adding R′SH(R′ may or may not be the same as R) in excess (at least equivalent in molar concentration to albumin and less than 1000 × the molar concentration). Adding an excess of R′SH converts the PEG-Alb(Cys-S—SR) to PEG-Alb (Cys-SH) and restores its antioxidant activity as well as amplify the antioxidant activity by means of the R′SH. For example, R′ might be glutathione or N-Acetyl Cysteine, both of which have well described antioxidant activities. We also may link albumin to many other medications using a very similar approach. In some cases, the medications will be peptides with an existing SH group (e.g., vasopressin) or will be peptides modified to have an available Cys group. In other cases, the medications will be non-peptides modified to react with the SH on Cys-34 such as Prostacyclin. The purpose of this linkage will be to:

a) Increase the circulating half life of the linked medication (e.g. erythropoietin);

b) Confine the effect of the linked medication to the vascular space (e.g. rapamycin); OR

c) Shield the medication from catabolic effects of certain enzymes leading to significant prolongation of its half-life e.g., Prostacyclin).

Other objects and advantages of the present invention will become apparent to those skilled in the art upon a review of the following detailed description of the preferred embodiments and the accompanying drawings.

### BRIEF DESCRIPTION OF THE DRAWINGS

FIG. 1 shows how Cys 34 preservation is effected, and the protecting agent is applied in excess maintaining the anti-oxidant effects of reduced Cys-34 as well as the protecting agent (e.g., Nacetyl Cysteine).

FIG. 2 shows purification of PEG-Albumin 5000ₙ on Q-Sepharose. PEG-Albumin 5000ₙ was prepared and applied to Q-Sepharose as described. Inset shows SDS gel electrophoresis of Q-Sepharose fractions: Alb, unmodified human albumin; U. unfractionated PEG-Albumin 5000ₙ, numbered lanes, samples from the corresponding fractions from Q-Sepharose.

### DETAILED DESCRIPTION OF THE INVENTION

The composition of this invention comprises a polyethylene glycol-albumin composition having at least one protected thiol region wherein the composition comprises PEG-Alb_{Cys-34} with Cys-34 preserved as a thiol. The albumin is linked to antioxidant medications with a reduced sulfhydryl group through the Cys 34 residing on the albumin. Preferably, the sulfhydryl group is represented by the formula (RSH). Preferably, the Cys 34 residing on albumin is represented by the formula Cys-S—SR. A compound represented by the formula R′SH is added to the composition to reduce the SH on Cys-34. R′ may or may not be the same as R. The compound represented by the formula R′SH is added in an amount ranging from at least equivalent in molar concentration to albumin to less than 1000 times the molar concentration of albumin. Preferably, the compound represented by the formula R′SH is added in an amount ranging from an excess in molar concentration to albumin to less than 1000 times the molar concentration of albumin. The compound represented by the formula R′SH converts PEG-Alb(Cys-S—SR) to PEG-Alb (Cys-SH). Preferably, R′ is glutathione or N-Acetyle Cysteine.

R and R^{I} are selected from the group consisting of hydrogen, cycloalkyl, alkyl, phenyl, and substituted phenyl. The substituted phenyl is substituted by halogen, alkyl, acryl, or alkoxy. The following terms used herein: “cycloalkyl”, “alkyl”, “acyl” and “alkoxy” generally contain from 1 to 50 carbons, as is well understood by those skilled in the art.

The halogens are five non-metallic elements found in group 17 of the periodic table. The Halogens are: fluorine, chlorine, bromine, iodine, and astatine.

Preferably R and R^{I} are hydrogen or an alkyl group containing 1 to 20 carbon atoms. Preferably, the halogen is chlorine. R^{I} may or may not be the same as R.

The volume-expanding properties of the PEG-albumin based colloid is a large albumin-based colloid composition which as a greater detail of hydration (13-16 times compared to albumin) and a larger hydrodynamic radius (3.4 nm vs. 10 nm). The PEG-albumin-based colloid composition is less likely to enter the extra vascular space than normal albumin. Additionally, the PEG-albumin-based colloid composition retains the important physiologic functions of albumin, including roles as an osmolyte, as an antioxidant, and as a transporter of less soluble metabolites such as heme and bilirubin; the latter two features are not associated with other crystalloids and colloids.

According to the present invention, unlike starches, the PEG-albumin composition retains the important physiologic functions of albumin, including roles as an osmolyte, as an antioxidant, and as a transporter of less soluble metabolities such as heme and bilirubin. The latter two features are not associated with other crystalloids and colloids. Protein unfolding studies performed on PEG-Alb indicated that albumin functionality is highly preserved. The albumin may be human albumin, bovine serum albumin, lactalbumin, or ovalbumin.

The albumin-based colloid composition is also useful as a hyperosmotic agent driving, or causing, ultra filtration inperitoneal dialysis. Still other uses include, for example, use in head trauma, hyperviscosity states, patients with liver cirrhosis following parcenthesis, Leukopheresis, nutritional albumin deficiency, nephrotric syndrome, liver failure, severe hypoalbuminemic patients, and severe burn patients.

In one aspect, the present invention comprises a composition of an albumin-based colloid composition having a preferred degree of hydration. The present invention further relates to two methods to produce the albumin-based colloid composition by modifying the albumin with polyethylene oxide: one is by using N-hydroxysuccinamide esters and the other is by using cyanuric chloride derivatives. The albumin-based colloid composition of the present invention is safe and has an extended useful half-life measured at least three times that of the normal albumin in normal rats and likely more prolonged in septic rats. The albumin-based colloid composition can be synthesized using recombinant albumin which decreases its immunogenicity.

The albumin-based colloid composition has a lessened tendency to extravascate because of its larger size, thereby avoiding worsening of the hypovolemic condition such as capillary leak syndrome and clinically, edema and compartment syndrome.

In another aspect, the volume-expanding properties of the albumin-based colloid (or example, albumin with covalently attached polyethylene glycol (PEG-Alb) is a large albumin-based colloid composition which has a greater degree of hydration (13-16 times compared to albumin) and a larger hydrodynamic radius (3.4 nm vs. 10 nm). The albumin-based colloid composition is less likely to enter the extra vascular space than normal albumin. Additionally, the albumin-based colloid composition retains the important physiologic functions of albumin, including roles as an osmolyte, as an antioxidant, and as a transporter of less soluble metabolites such as heme and bilirubin; the latter two features are not associated with other crystalloids and colloids.

In one aspect, the present invention relates to a composition comprising a large albumin-based colloid with a preferred degree of hydration. The composition is an albumin-based colloid and, in one embodiment, comprises a polyethylene glycol modified albumin having a hydrodynamic radius sufficiently large to preclude the molecule from leaking through a patient's capillaries. In certain embodiments, the albumin-based colloid composition has an average molecular weight of at least 128.000 daltons. The composition can comprise human albumin, bovine serum albumin, lactalbumin, or ovalbumin.

The albumin-based colloid composition has an ability to bind ligands such as sodium ions, fatty acids, bilirubin and therapeutic drugs.

In another aspect, the present invention relates to an in vivo method of preventing or treating hypovolemic conditions and its complications such as multiple organ dysfunction syndrome comprising administering a therapeutic amount of the large albumin-based colloid composition to a patient in danger of developing such conditions.

In another aspect, the present invention relates to a method for the prevention of mammalian tissue injured or at risk of injury comprising the administration of a therapeutic amount to a mammal of a composition comprising an albumin-based colloid. Example solid organs preservation before transplantation. The composition is incapable of leaking through the mammal's capillaries and is present in an amount of sufficient to protect the tissue from injury. The method is especially useful where the risk of injury is due to hypovolemia, sepsis, shock, burn, trauma, surgery, predisposition to capillary leak, hyperviscosity stress, hypoalbuminemia, and/or anoxia.

As a result, the compositions of this invention may be used in an in vivo method of treating a patient by increasing the circulating half life of the linked medication (e.g., erythropoietin). The composition also may be used in treating mammalian tissue injury by the administration of a therapeutic amount to a mammal of the composition. This method further comprises the step of confining the linked medication to the vascular space (e.g., rapamycin). An in vivo method of treating conditions in a patient further comprises the step of shielding the medication from catabolic effects of certain enzymes. This leads to significant prolongation of its half-life (e.g. Prostacylcin).

Other antioxidants include the following. An antioxidant is a molecule capable of slowing or preventing the oxidation of other molecules. Oxidation is a chemical reaction that transfers electrons from a substance to an oxidizing agent. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents such as thiols or polyphenols.

Antioxidants are classified into two broad divisions, depending on whether they are soluble in water (hydrophilic) or in lipid (hydrophobic). In general, water-soluble antioxidants react with oxidants in the cell cytosol and the blood plasma, while lipid-soluble antioxidants protect cell membranes from lipid peroxidation. These compounds may be synthesized in the body or obtained from the diet. The different antioxidants are present at a wide range of concentrations in body fluids and tissues, with some such as glutathione or ubiquinione mostly present within cells, while others such as uric acid are more evenly distributed.

The following table shows the properties of some common antioxidants.

| | | | Concentration |
|---|---|---|---|
| Antioxidant | | Concentration in human | in liver tissue |
| Metabolite | Solubility | serum (μM) | (μmol/kg) |
| Ascorbic acid | Water | 50-60 | 260 (human) |
| (Vitamin C) | | | |
| Glutathione | Water | 325-650 | 6,400 (human) |
| Lipoic acid | Water | 0.1-0.7 | 4-5 (rat) |
| Uric Acid | Water | 200-400 | 1,600 (human) |
| Carotenes | Lipid | β-carotene: 0.5-1 | 5 (human, total |
| | | retinol (vitamin A): 1-3 | carotenoids) |
| A-Tocopherol | Lipid | 10-40 | 50 (human) |
| (vitamin E) | | | |
| Ubiquinol | Lipid | 5 | 200 (human) |
| (coenzyme Q) | | | |

In another embodiment, gadolinium may be linked to the PEG Alb platform of this invention. Gadolinium is a chemical element that has the symbol Gd and atomic number 64. It is a silvery-white, malleable and ductile rare-earth metal. Gadolinium has exceptionally high absorption of neutrons and therefore is used for shielding in neutron radiography and in nuclear reactors. Because of its paramagnetic properties, solutions of organic gadolinium complexes and gadolinium compounds are the most popular intravenous MRI contrast agents in medical magnetic resonance imaging.

The following examples are provided merely to further illustrate the present invention. The scope of the invention shall not be construed as merely consisting of the following examples.

### Example I

### Preparation of PEG-Albumin 5000ₙ

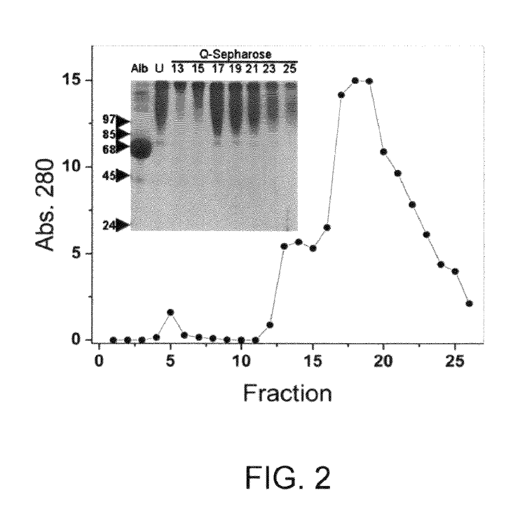

This form of PEG-modified albumin is modified at multiple lysyl residues is follows. There are some modifications in the purification (ion exchange chromatography in place of gel filtration) to allow for more practical scale up. Methoxypolyethylene glycol cyanuric chloride (average Mr 5000) is added with gentle stirring to human albumin (Cohn fraction V) dissolved in 10 mM potassium phosphate buffer (pH 7.5) at 50-60 mg/ml; four additions (0.2 g/g of albumin) are made at 10 min intervals at 22° C. The reaction is stirred 40 min after the last addition of reagent. Modification is rapid, being complete in less than 15 min at room temperature with the extent of modification depending primarily on the amount of reagent added. PEG-Alb is applied to Q-Sepharose (1 ml of resin per 0.025 grams of albumin) equilibrated with 10 mM potassium phosphate buffer (pH 7.4), washed with three column volumes of starting buffer and eluted with 0.25 M NaCl. Excess unincorporated PEG reagent elutes in the unbound fraction and PEG-albumin is concentrated in an Amicon ultrafiltration cell employing a PM10 membrane (Millipore) and dialyzed against 20 volumes of 0.15 M NaCl at 4° C. for 20 hours with one change of 0.15 M NaCl. This material is free of unmodified albumin and unreacted PEG5000. An elution profile for PEG-Albumin 5000ₙ is shown in FIG. 2. Other amine selective PEG reagents that can be employed using this protocol include: N-hydroxy succinimide esters; aldhydes (with reduction of the Schiffs formed with the ε-amino group of lysyl residues: p-nitrophenyl esters.

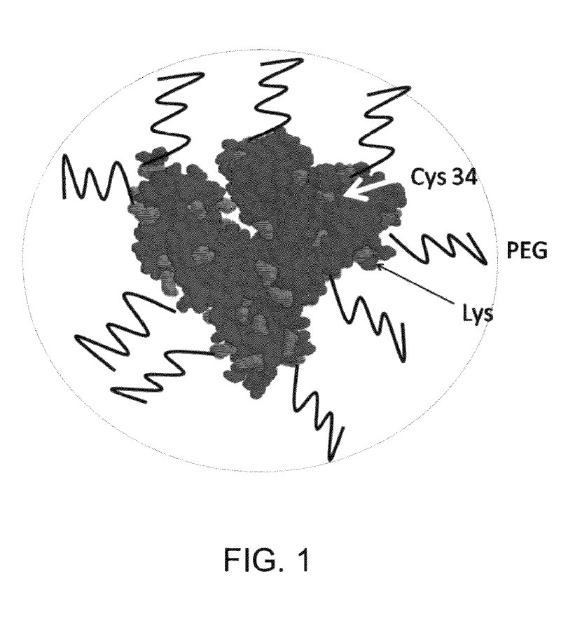

FIG. 1 shows how Cys 34 preservation is effected, and the protecting agent is applied in excess maintaining the anti-oxidant effects of reduced Cys-34 as well as the protecting agent (e.g., Ncetyl Cysteine).

### Example II

### Production of Peg-Albumin with Protection of Cys-34

Albumin in 10 mM potassium phosphate buffer (pH 7.5) at 50-60 mg/ml is incubated with a two to three-fold molar excess over protein thiol (2.5 mM) of DTNB (5,5′-dithiobis 2-nitrobenzoate), for 30 minutes at 22° C. The preparation is then modified with methoxypolyethylene glycol cyanuric chloride as described above for PEG-albumin. Dithiothreitol or tris(2-carbocymethyl)-phosphine is added to the preparation to 4 mM and incubated for 1 hr at 22° C. PEG-albumin is purified by ion exchange chromatography, concentrated by ultrafiltration and dialyzed as described above for PEG-albumin.

FIG. 2 shows purification of PEG-Albumin 5000n on Q-Sepharose. PEG-Albumin 5000ₙ was prepared and applied to Q-Sepharose as described. Inset shows SDS gel electrophoresis of Q-Sepharose fractions: Alb, unmodified human albumin; U. unfractionated PEG-Albumin 5000ₙ, numbered lanes, samples from the corresponding fractions from Q-Sepharose.

### Example III

Thiol selective reagents—Modification through a thiol is a useful approach for human serum albumin since it has a single thiol (cys34). Human serum albumin is a mixture of protein with cys34 as a free thiol and a substantial fraction with the thiol modified with glutathione or as a disulfide dimmer of two albumins. Under mild conditions, Cys34 disulfides can be reduced such that all of the cys34 is available as a free thiol without reduction of the less accessible disulfides. Cys34 is reactive with thiol selective reagents, including N-ethylmaleimide and iodoacetamide. Albumin is modified with mPEG-maleimide derivatives such that the PEG is linked to a single site on the protein. Modification at a single, unique site is less likely to perturb native structure or alter the ligand binding properties of the albumin. As indicated in the preliminary results section, we have prepared two such forms of mPEG-Alb. A potential disadvantage of thiol modification is that it may alter the antioxidant properties of the product.

### PEG Derivatives of Different Sizes and Geometries

Albumins modified with different sizes PEGs and PEGs with branched structures are examined. Sizes available include 3,400 Mᵣ, 5,000 Mᵣ, 20,000 Mᵣ, and 40,000 Mᵣ. There are branched (3 in FIG. 29) and forked (5 in FIG. 29) versions of PEG with various chemistries for linkage to proteins (46a, 117a). Larger PEGs allow for modification at fewer sites to achieve the same effective size. The larger size distribution is particularly important for linkage through cys34 since there is only one PEG incorporated. A consideration relating to reagent size is that smaller PEG-peptides (e.g. PEG≦1200 (119) are readily cleared through the kidneys, justifying analysis of multiply modified albumin. Increasing PEG chain length prolongs the half-life of the material in the circulation.

Preservation of Cys 34—The activity of albumin in inhibiting apoptosis and other biological properties depend on thiols (cys34). MPEG-Albs that retain cys 34 as a thiol are prepared. Albumin is treated with a slight excess of dithiothreitol followed by modification of cys 34 with 5,5′-dithiobis-2-nitrobenzoic acid. Low molecular weight products are removed by gel filtration and the protein is modified with an amine selective PEG reagent. The free thiol is regenerated by treating the protein with dithiothreitol to release the thionitrobenzoic acid (monitored spectrally at 412 nm). The mPEG albumin is purified to remove unmodified protein, excess reagent and reaction byproducts. The MPEG-albumins produced using this approach are modified at multiple sites since the reagents modify lysyl residues. However, it is also within the contemplated scope that the method can include using larger PEG reagents (e.g., PEG20000 and PEG40000) the number of residues modified can be minimized by varying reagent concentration and reaction conditions.

### Example IV

### Antioxidant Activity

Ischemia-reperfusion results in disrupting endothelial integrity. When pulmonary artery endothelial cells (EC) were exposed to ischemic human plasma, ten minutes later they became rounded, formed gaps and then blebbed. The same morphologic changes occurred in microdermal EC culture after exposure to sera from capillary leak syndrome patients. Apoptosis of EC was evidenced by morphologic criteria, plasma phosphatidylserine exposure (Annexin staining), and DNA fragmentation. Increased Bax/Bcl2 in endothelial cells was detected by immunohistochemistry. The mechanism of these effects was explored by measuring intracellular reactive oxygen species (ROS) and the results suggested that oxidative injury played a role in the mechanism of EC apoptosis. Oxidative stress is a well known inducer of apoptosis. In addition increased apoptosis occurs after trauma and hemorrhage. Inhibition of apoptosis by caspase inhibitors attenuated I/R induced inflammation. In tissues exposed to ischemia-reperfusion, antioxidants minimized the damage from this injury. Albumin is the major extracellular antioxidant in plasma. It exerts this function through the enzyme gamma glutamylcysteine dipeptide, where albumin plays a significant role in glutathione synthesis. Glutathione is the main low molecular weight soluble thiol present in mammalian cells, its depletion plays a role in the induction of apoptosis. In another study looking at how albumin exerts its antioxidant activity, modification of the single free thiol (cys 34) was accompanied by a 45% decrease in antioxidant activity. Albumin is protected against oxidation by its capacity to increase glutathione (GSH). Conversely, reduction in GSH led to a) activation of caspase 3 and poly ADP ribose polymerase (PARP) fragmentation and b) the decrease in Bcl-2/Bax ratio. The latter ratio is a strong indicator of cell survival, particularly in defense against oxidative injury. As a result, albumin, through its function as antioxidant, contributes significantly to the protective effect against apoptosis. In reference to the endothelium, albumin reduced microvascular permeability and played an essential role in preventing apoptosis of endothelial cells.

While this invention has been described with emphasis upon preferred embodiments, it would be obvious to those of ordinary skill in the art that preferred embodiments may be varied. It is intended that the invention may be practiced otherwise than as specifically described herein. Accordingly, this invention includes all modifications and claims spirit and scope of the appended claims.

We claim:

1. A composition comprising a polyethylene glycol-albumin composition having at least one protected thiol region wherein the composition comprises PEG-Alb_{Cys-34} with Cys-34 preserved as a thiol, wherein the albumin is linked to antioxidant medications with a reduced sulfhydryl group through the Cys 34 residing on the albumin.

2. A composition according to claim 1 wherein the medications are antioxidants.

3. A composition according to claim 1 wherein the sulfhydryl group is represented by the formula (RSH).

4. A composition according to claim 1 wherein the Cys 34 residing on albumin is represented by the formula Cys-S—SR.

5. A composition according to claim 3 wherein a compound represented by the formula R′SH is added to the composition to reduce the SH on Cys-34.

6. A composition according to claim 5 wherein R′ may or may not be the same as R.

7. A composition according to claim 5 wherein the compound represented by the formula R′SH is added in an amount ranging from at least equivalent in molar concentration to albumin to less than 1000 times the molar concentration of albumin.

8. A composition according to claim 5 wherein the compound represented by the formula R′SH is added in an amount ranging from an excess in molar concentration to albumin to less than 1000 times the molar concentration of albumin.

9. A composition according to claim 5 wherein the compound represented by the formula R′SH converts PEG-Alb(Cys-S—SR) to PEG-Alb (Cys-SH).

10. A composition according to claim 5 wherein R′ is glutathione.

11. A composition according to claim 5 wherein R′ is N-Acetyle Cysteine.

12. A composition according to claim 1 wherein the medication is gadolinium.

13. A composition comprising a polyethylene glycol-albumin composition having at least one protected thiol region wherein the composition comprises PEG-Alb_{Cys-34} preserved as a thiol, wherein the albumin is linked to medications through the Cys 34 residing on the albumin.

14. The composition of claim 13, wherein the albumin is human albumin, bovine serum albumin, lactalbumin, or ovalbumin.

15. An in vivo method of treating conditions in a comprising administering a therapeutic amount of the composition of claim 13 to increase the circulating half life of the linked medication.

16. A method according to claim 15 wherein the linked medication is erythropoietin.

17. A method for treating mammalian tissue injury comprising the administration of a therapeutic amount to a mammal of the composition of claim 13.

18. A method according to claim 17 further comprising the steps of confining the linked medication to the vascular space.

19. A method according to claim 18 wherein the linked medication is rapamycin.

20. An in vivo method of treating conditions in a patient comprising administering a therapeutic amount of the composition of claim 13 to shield the linked medication from catabolic effects of enzymes.

21. The method of claim 20 further comprising the step of significant prolongation of the half-life of the linked medication.

22. The method of claim 21 wherein the linked medication is Prostacyclin.

23. The method of claim 21 wherein the linked medication is an antioxidant.

24. The method of claim 21 wherein the linked medication is gadolinium.
